SEQUENCE LISTING (18 sequences)

SEQ ID NO: 1 - DNA, 19 bp, Mus baoulei
  source 1..19: mol_type = genomic DNA
gccagactagcatctggga

SEQ ID NO: 2 - DNA, 19 bp, Mus baoulei
  source 1..19: mol_type = genomic DNA
tcctctttagaagccacca

SEQ ID NO: 3 - DNA, 20 bp, Mus baoulei
  source 1..20: mol_type = genomic DNA
ggaaggtataaaagcccttc

SEQ ID NO: 4 - DNA, 19 bp, Homo sapiens
  source 1..19: mol_type = genomic DNA
tcttcttcagactgcgcca

SEQ ID NO: 5 - DNA, 20 bp, Homo sapiens
  source 1..20: mol_type = genomic DNA
agagcaagtatgggctcact

SEQ ID NO: 6 - DNA, 20 bp, Homo sapiens
  source 1..20: mol_type = genomic DNA
tcttcttcagactgcgccat

SEQ ID NO: 7 - DNA, 20 bp, Homo sapiens
  source 1..20: mol_type = genomic DNA
cttcttcagactgcgccatg

SEQ ID NO: 8 - DNA, 21 bp, Homo sapiens
  source 1..21: mol_type = genomic DNA
ggtcttcttcagactgcgcca

SEQ ID NO: 9 - DNA, 19 bp, Homo sapiens
  source 1..19: mol_type = genomic DNA
aaggtataaaaacgccctt

SEQ ID NO: 10 - DNA, 20 bp, Mus baoulei
  source 1..20: mol_type = genomic DNA
agtccccagcaggatcacat

SEQ ID NO: 11 - DNA, 19 bp, Mus baoulei
  source 1..19: mol_type = genomic DNA
tctgctcgcagggtcccaa

SEQ ID NO: 12 - DNA, 20 bp, Mus baoulei
  source 1..20: mol_type = genomic DNA
gcaaggctgaggttcacagg

SEQ ID NO: 13 - DNA, 19 bp, Homo sapiens
  source 1..19: mol_type = genomic DNA
gtgttaccgaatggcatgg

SEQ ID NO: 14 - DNA, 19 bp, Homo sapiens
  source 1..19: mol_type = genomic DNA
ttaccgaatggcatggtgg

SEQ ID NO: 15 - DNA, 19 bp, Homo sapiens
  source 1..19: mol_type = genomic DNA
gggaagattgaagattagc

SEQ ID NO: 16 - DNA, 20 bp, Homo sapiens
  source 1..20: mol_type = genomic DNA
ggtgttaccgaatggcatgg

SEQ ID NO: 17 - DNA, 20 bp, Homo sapiens
  source 1..20: mol_type = genomic DNA
tacaccgccaccatgccatt

SEQ ID NO: 18 - DNA, 19 bp, Homo sapiens
  source 1..19: mol_type = genomic DNA
gcggtgtaggagacctgat

(19) United States
(12) Patent Application Publication
(10) Pub. No.: US 2024/0384305 A1
(43) Pub. Date: Nov. 21, 2024

(54) IDENTIFICATION AND VALIDATION OF GENOMIC SAFE-HARBOR SITES IN SKELETAL MUSCLE FOR TARGETED INTEGRATION

(71) Applicant: BOARD OF TRUSTEES OF THE UNIVERSITY OF ARKANSAS, Little Rock, AR (US)

(72) Inventors: Made Harumi Padmaswari, Fayetteville, AR (US); Christopher Nelson, Springdale, AR (US)

(73) Assignee: BOARD OF TRUSTEES OF THE UNIVERSITY OF ARKANSAS, Little Rock, AR (US)

(21) Appl. No.: 18/665,417

(22) Filed: May 15, 2024

Related U.S. Application Data
(60) Provisional application No. 63/466,566, filed on May 15, 2023.

(51) Int. Cl. C12N 15/90; C12N 9/22; C12N 15/11

(52) U.S. Cl. CPC C12N 15/907; C12N 9/22; C12N 15/11; C12N 2310/20

(57) ABSTRACT

Embodiments of the present disclosure pertain to a method of expressing a nucleic acid sequence in a cell by inserting the nucleic acid sequence at a region of a creatine kinase (CKM) and/or a myoglobin (MB) gene such that the inserted nucleic acid sequence becomes expressed in the cell by a promoter of the gene. The nucleic acid sequences may be expressed in vitro or in vivo for various purposes, such as treatment or prevention of a disease and/or tissue repair or regeneration. Additional embodiments pertain to a system for expressing a nucleic acid sequence in a cell after insertion at a region of a creatine kinase (CKM) gene and/or a myoglobin (MB) gene. Further embodiments of the present disclosure pertain to a modified cell that includes an inserted nucleic acid sequence at a region of a creatine kinase (CKM) gene and/or a myoglobin (MB) gene.

### CROSS-REFERENCE TO RELATED APPLICATIONS

This application claims priority to U.S. Provisional Patent Application No. 63/466,566, filed on May 15, 2023. The entirety of the aforementioned application is incorporated herein by reference.

### STATEMENT REGARDING FEDERALLY SPONSORED RESEARCH

This invention was made with government support under R00 EB023979 awarded by the National Institutes of Health. The government has certain rights in the invention.

### SEQUENCE DISCLOSURE STATEMENT

Pursuant to 37 C.F.R. § 1.834, Applicant has submitted a sequence listing in XML format (“Sequence Listing”). The name of the file containing the Sequence Listing is “AF42186.P031US”. The date of the creation of the Sequence Listing is Jul. 24, 2024. The size of the Sequence Listing is 17,000 bytes. Applicant hereby incorporates by reference the material in the Sequence Listing.

### BACKGROUND

Current methods and systems for expressing nucleic acids in cells have numerous limitations. Embodiments of the present disclosure aim to address these limitations.

### SUMMARY

In some embodiments, the present disclosure pertains to a method of expressing a nucleic acid sequence in a cell. In some embodiments, the methods of the present disclosure include a step of inserting the nucleic acid sequence at a region of a creatine kinase (CKM) gene and/or a myoglobin (MB) gene. Thereafter, the inserted nucleic acid sequence may become expressed in the cell by a promoter of the gene. In some embodiments, the nucleic acid sequences of the present disclosure may be expressed in vitro. In some embodiments, the nucleic acid sequences of the present disclosure may be expressed in vivo for various purposes, such as treatment or prevention of a disease and/or tissue repair or regeneration.

Additional embodiments of the present disclosure pertain to a system for expressing a nucleic acid sequence in a cell. In some embodiments, the systems of the present disclosure include a nucleic acid sequence operational for insertion at a region of a creatine kinase (CKM) gene and/or a myoglobin (MB) gene. In some embodiments, the inserted nucleic acid sequence is operational to become expressed in the cell by a promoter of the gene.

Further embodiments of the present disclosure pertain to a modified cell that includes an inserted nucleic acid sequence at a region of a creatine kinase (CKM) gene and/or a myoglobin (MB) gene. In some embodiments, the inserted nucleic acid sequence becomes expressed in the cell by a promoter of the gene.

### DRAWINGS

FIG. 1 illustrates a method of expressing a nucleic acid in a cell in accordance with various embodiments of the present disclosure.

FIG. 2 provides a schematic representation of ideal safe-harbor sites. The bar in the middle represents the optimal location of safe-harbor sites. The site should preferably be unique and located in open chromatin. Within 300 kb, the site should preferably be free from any cancer-related genes, miRNAs, or other functional small RNAs. Within 50 kb, the site should preferably be free from the 5′ end of a gene, replication origin, and ultra-conserved elements. Additionally, the site should preferably be in a region of low transcriptional activity or have no mRNA within 25 kb.

FIGS. 3A-3F illustrate targeted integration of a promoter-less green fluorescent protein (GFP) at myoglobin (Mb) and creatine kinase (CKM), which leads to GFP expression in myotubes. FIG. 3A (top portion) provides an overview of transgene integration to hijack endogenous Ckm or Mb promoters. The homology-independent targeted integration (HITI)-based gene editing system is delivered via plasmid transfection, which encodes the CMV-driven S. pyogenes Cas9 protein, U6 promoter-gRNA expression cassette, and a donor plasmid containing a transgene fragment flanked by Cas9 target sites in the reverse orientation relative to genomic DNA. FIG. 3B (bottom portion) shows the schematic mechanism at the molecular level, which shows that, by integrating the transgene in the intronic region, only correct integration is expected to produce the protein, while partial or reverse integration is expected to not affect endogenous genes. The figure is not drawn to scale. FIG. 3B shows the gRNA target site map in Ckm and Mb locus. Five gRNA target sites were selected within 250 bp in the splice acceptor and 5′ UTR region of the gene. FIG. 3C shows the quantification of gRNA efficiency in C2C12 cell lines with short-read next-generation sequencing (NGS) based on indel percentage. The error bars were computed as the standard deviation obtained through bootstrap resampling technique. FIG. 3D shows validation of correct GFP integration in genomic DNA, which is shown in both 5′ and 3′ integration regions in the treated group. The schematic figure shows the forward primer in the GFP transgene overlaps with the reverse primer (Primer list). FIG. 3E shows validation of correct mRNA splicing from GFP integration by cDNA PCR, which is shown in both loci. At the transcriptome level, only 5′ integration was assessed. FIG. 3F shows fluorescent microscopy images assessing GFP expression in edited myotubes at 10 days after editing. The scale bar is 50 μm.

FIGS. 4A-4E shows that targeted integration of a promoter-less hF9 gene leads to sustained expression. FIG. 4A shows a schematic of the structure of HITI insert plasmid for human F9 expression. Full-length cDNA region from exon 1 to exon 8 is cloned to replace GFP transgene in the previous construct. FIGS. 4B and 4C show genotyping of the F9 integration in C2C12 cell line using primers spanning the junction between the integration site and the transgene in genomic DNA and cDNA. FIG. 4D shows relative hF9 expression following plasmid transfection. Five biological independent samples, mean±s.e.m. All samples were processed at 10 days post-transfection including 7 days of myotubes differentiation. FIG. 4E shows time course cell culture to assess the expression of hF9 in treated C2C12 for 30 days without selection compared to the scrambled group. Two independent biological replicates, means±sem.

FIGS. 5A-5F show deep sequencing results, which reveal precise and imprecise outcomes of targeted integration. FIGS. 5A-5B show deep sequencing results, which show a varied range of precise integration in 5′ and 3′ junctions. A similar pattern was observed in both 5′ and 3′0 junctions in both loci, vector chewback that predominantly happened on the insert side. FIG. 5C shows deep sequencing at the mRNA level, which shows a higher percentage of precise integration in three biological replicates in the treated Ckm locus. FIG. 5D shows modification frequency trace from the fusion of Ckm and GFP transgene. FIG. 5E (top) shows a schematic Tn5 tagmentation-based sequencing to quantify integration efficiency. FIG. 5E (bottom) shows the graph of a percentage of reads aligned to the Ckm gene and reference amplicon. FIG. 5F shows characterization of the fusion of Ckm and hF9 cDNA using 5′RACE with GSP reverse transcriptase primer.

FIGS. 6A-6D show RNA sequencing results, which reveal significant increases in transgene expression and relative precision of integration at Ckm over Mb. FIG. 6A shows a pipeline for bulk RNA-seq experiment on Ckm- and Mb-integrated and scrambled non-integrated C2C12 myotube cells. FIG. 6B shows principal component analysis of two biological replicates of C2C12 myotubes with Ckm, Mb, or scrambled treatment. FIG. 6C shows hF9 transcript expression level measured in TPM (mean±sem). FIG. 6D shows differential expression of genes following hF9 integration in Ckm and Mb loci.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The current wave of therapies transforming genetic medicines rely on gene replacement. Efficient transgene expression is predominantly achieved through the use of adeno-associated viral (AAV) vectors to express a therapeutic gene under an exogenous promoter. While multiple studies have reported the sustainability of AAV gene therapy for years after a single administration, others have noted a decline in transgene expression, with some even observing a return to baseline levels.

With the majority of transgene expression sustained episomally, research indicates that epigenetic silencing of episomal DNA and interactions between the AAV capsid and host factors significantly contribute to the reduction in expression. In line with these findings, recent studies in dogs and non-human primates revealed initially high but short-lived expression from episomal genomes, while long-term expression of the transgene was attributed to clonal expansion of cells containing integrated vectors.

However, transgene integration in AAV gene replacement happens in a semi-random pattern throughout the genome. Vector integration in unpredictable positions in the genome might result in unforeseeable expression and interactions with the host genome, though no adverse events related to vector integration have been detected in humans. Thus, these limitations highlight the need to transition from random integration with viral vectors to targeted, site-specific methods such as clustered regularly interspaced short palindromic repeats (CRISPR)-Cas technology, which can overcome the problem of insertion of correct genes into random genomic sites.

CRISPR-based integration methods have evolved significantly, from using the endogenous repair pathways, non-homologous end joining (NHEJ) or homology-directed repair (HDR), to engineered transposable elements. These gene editing technologies emerged as a potential avenue for long-term therapeutic transgene insertion.

Despite considerable advancements in targeted gene integration methods, identifying the locus to insert replacement genes for optimal safety and efficiency remains challenging. While many gene editing approaches to target the disease locus itself, some studies indicate that the genomic structure of mutated genes may not favor entire gene replacement. Further, some exogenous promoters, such as cytomegalovirus (CMV) and human elongation factor-1 alpha (EF1alpha), are susceptible to promoter silencing. Therefore, exploring loci with high transcriptional activity emerges as an alternative.

The current strategy of “hijacking” the endogenous promoter of high transcriptional activity genes or transgene overexpression remains largely organ-specific. While the results are promising, the safety and versatility of this approach have yet to undergo thorough investigation. Ensuring the safety and versatility of these sites is desirable for achieving stable expression of integrated transgenes without adversely affecting the host cell.

Empirical studies have identified safe-harbor sites that support long-term transgene expression. There are three well-established sites: AAVS1, CCR5, and hROSA26, and recently explored sites such as Rogil, Rogi2, or SHS231. However, the expression from these sites is limited or tissue-specific and requires the inserted vector to include an exogenous promoter.

As such, a need exists for more effective methods and systems for expressing nucleic acids in cells. Numerous embodiments of the present disclosure aim to address the aforementioned need.

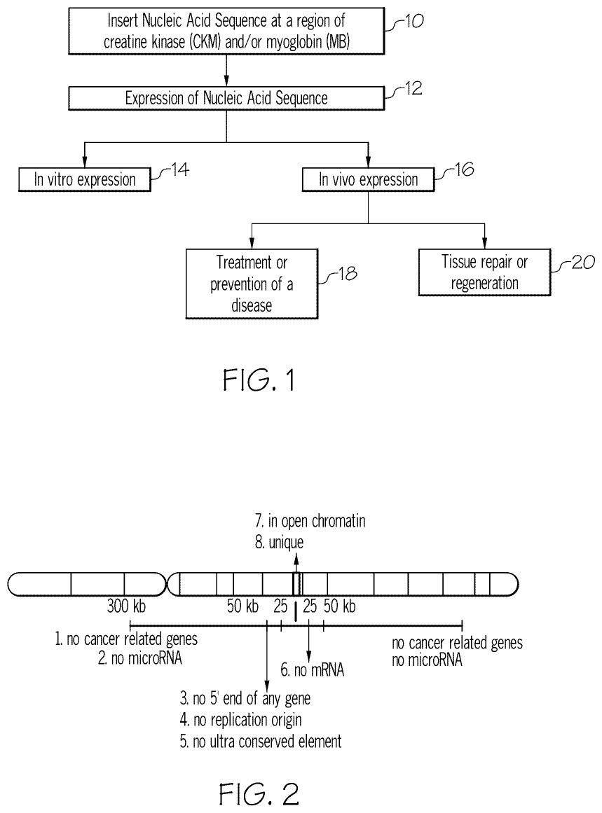

In some embodiments, the present disclosure pertains to a method of expressing a nucleic acid sequence in a cell. In some embodiments illustrated in FIG. 1, the methods of the present disclosure include inserting the nucleic acid sequence at a region of a creatine kinase (CKM) gene and/or a myoglobin (MB) gene (step 10). Thereafter, the inserted nucleic acid becomes expressed in the cell (step 12). For instance, in some embodiments, the inserted nucleic acid sequence may become expressed in the cell by a promoter of a gene. In some embodiments, the nucleic acid sequences of the present disclosure may be expressed in vitro (step 14). In some embodiments, the nucleic acid sequences of the present disclosure may be expressed in vivo (step 16) for various purposes, such as treatment or prevention of a disease (step 18) and/or tissue repair or regeneration (step 20).

Additional embodiments of the present disclosure pertain to a system for expressing a nucleic acid sequence in a cell. In some embodiments, the systems of the present disclosure include a nucleic acid sequence operational for insertion at a region of a creatine kinase (CKM) gene and/or a myoglobin (MB) gene. In some embodiments, the inserted nucleic acid sequence is operational to become expressed in the cell by a promoter of the gene.

Further embodiments of the present disclosure pertain to a modified cell that includes an inserted nucleic acid sequence at a region of a creatine kinase (CKM) gene and/or a myoglobin (MB) gene. In some embodiments, the inserted nucleic acid sequence becomes expressed in the cell by a promoter of the gene.

For the purposes of this application, CKM and MB may refer to genes from different species, such humans, mice, and other species. As set forth in more detail herein, the methods, systems and modified cells of the present disclosure can have numerous embodiments. Additionally, the methods, systems and modified cells of the present disclosure can have numerous applications.

### Genes

The nucleic acid sequences of the present disclosure may be inserted into regions of various genes. For instance, in some embodiments, the gene is an MB gene. In some embodiments, the gene is a human version of the MB gene. In some embodiments, the gene is a non-human version of the MB gene. In some embodiments, the gene is a murine version of the MB gene.

In some embodiments, the gene is a CKM gene. In some embodiments, the gene is a human version of the CKM gene. In some embodiments, the gene is a non-human version of the CKM gene. In some embodiments, the gene is a murine version of the CKM gene.

The nucleic acid sequences of the present disclosure may be inserted into various regions of CKM and/or MB genes. For instance, in some embodiments, the region is positioned at an intron region of the gene, an exon region of the gene, inside of the gene, an untranslated region (UTR) of the gene, a 5′ untranslated region (UTR) of the gene, a 3′ untranslated region (UTR) of the gene, an intergenic region of the gene, a genomic safe harbor site of the gene, a promoter of the gene, a region operable to be expressed by the promoter of the gene, or combinations thereof. In some embodiments, the region is positioned at an intron region of the gene.

In some embodiments, the nucleic acid sequences of the present disclosure may be inserted into a region of MB. In some embodiments, the region includes a region operable to be expressed by the promoter of MB. In some embodiments, the region is located at an intron of MB. In some embodiments, the region is located at intron 1 of MB. In some embodiments, the MB region includes a sequence that includes, without limitation, GCCAGACTAGCATCTGGGA (SEQ ID NO: 1), TCCTCTTTAGAAGCCACCA (SEQ ID NO: 2), GGAAGGTATAAAAGCCCTTC (SEQ ID NO: 3), TCTTCTTCAGACTGCGCCA (SEQ ID NO: 4), AGAGCAAGTATGGGCTCACT (SEQ ID NO: 5), TCTTCTTCAGACTGCGCCAT (SEQ ID NO: 6), CTTCTTCAGACTGCGCCATG (SEQ ID NO: 7), GGTCTTCTTCAGACTGCGCCA (SEQ ID NO: 8), AAGGTATAAAAACGCCCTT (SEQ ID NO: 9), a sequence with at least 65% sequence identity with any one of SEQ ID NOS: 1-9, a sequence with at least 70% sequence identity with any one of SEQ ID NOS: 1-9, a sequence with at least 75% sequence identity with any one of SEQ ID NOS: 1-9, a sequence with at least 80% sequence identity with any one of SEQ ID NOS: 1-9, a sequence with at least 85% sequence identity with any one of SEQ ID NOS: 1-9, a sequence with at least 90% sequence identity with any one of SEQ ID NOS: 1-9, a sequence with at least 95% sequence identity with any one of SEQ ID NOS: 1-9, a sequence with at least 99% sequence identity with any one of SEQ ID NOS: 1-9, or combinations thereof.

In some embodiments, the nucleic acid sequences of the present disclosure may be inserted into a region of CKM. In some embodiments, the region includes a region operable to be expressed by the promoter of CKM. In some embodiments, the region is located at an intron of CKM. In some embodiments, the region is located at intron 1 of CKM. In some embodiments, the CKM region includes a sequence that includes, without limitation, AGTCCCCAGCAGGATCACAT (SEQ ID NO: 10), TCTGCTCGCAGGGTCCCAA (SEQ ID NO: 11), GCAAGGCTGAGGTTCACAGG (SEQ ID NO: 12), GTGTTACCGAATGGCATGG (SEQ ID NO: 13), TTACCGAATGGCATGGTGG (SEQ ID NO: 14), GGGAAGATTGAAGATTAGC (SEQ ID NO: 15), GGTGTTACCGAATGGCATGG (SEQ ID NO: 16), TACACCGCCACCATGCCATT (SEQ ID NO: 17), GCGGTGTAGGAGACCTGAT (SEQ ID NO: 18), a sequence with at least 65% sequence identity with any one of SEQ ID NOS: 10-18, a sequence with at least 70% sequence identity with any one of SEQ ID NOS: 10-18, a sequence with at least 75% sequence identity with any one of SEQ ID NOS: 10-18, a sequence with at least 80% sequence identity with any one of SEQ ID NOS: 10-18, a sequence with at least 85% sequence identity with any one of SEQ ID NOS: 10-18, a sequence with at least 90% sequence identity with any one of SEQ ID NOS: 10-18, a sequence with at least 95% sequence identity with any one of SEQ ID NOS: 10-18, a sequence with at least 99% sequence identity with any one of SEQ ID NOS: 10-18, or combinations thereof.

### Insertion of Nucleic Acid Sequences

The present disclosure may utilize various methods to insert a nucleic acid sequence at a region of a gene. Additionally, the nucleic acid sequences of the present disclosure may be operational for insertion at a region of a gene through various methods. For instance, in some embodiments, nucleic acid sequence insertion occurs by a method that includes, without limitation, site-specific nucleic acid integration, site-specific recombination, RNA-guided nucleic acid integration, insertion by a gene editing system, insertion by a clustered regularly interspaced short palindromic repeats (CRISPR)/nuclease system (CRISPR system), insertion by non-DSB (double strand break) CRISPR system with integrase or transposase combination, homology-independent targeted integration (HITI), or combinations thereof.

In some embodiments, the insertion of a nucleic acid sequence at a region of a gene occurs through the utilization of a clustered regularly interspaced short palindromic repeats (CRISPR)/nuclease system (CRISPR system). In some embodiments, the systems of the present disclosure include a CRISPR system. In some embodiments, the insertion of the nucleic acid sequence includes introducing the nucleic acid sequence and the CRISPR system to the cell, where the CRISPR system includes a guide RNA that directs the nuclease of the CRISPR system to a region of a gene, where the nuclease cuts at the region of the gene, and where the nucleic acid sequence is inserted at the cut region of the gene for expression.

The CRISPR systems of the present disclosure may include various nucleases. For instance, in some embodiments, the nuclease of the CRISPR system includes, without limitation, Cas nucleases, class 2 of Cas nucleases, Cas 9, Cas Φ, CasΦ2, Cas12a, non-CRISPR enzymes, meganucleases, zinc finger nucleases, talens, or combinations thereof. In some embodiments, the nuclease includes Cas 9.

The CRISPR systems of the present disclosure may also utilize various guide RNAs. For instance, in some embodiments, the guide RNAs of the CRISPR system direct the nuclease to a region of CKM. In some of such embodiments, such guide RNAs direct the nuclease to a CKM region that includes, without limitation, AGTCCCCAGCAGGATCACAT (SEQ ID NO: 10), TCTGCTCGCAGGGTCCCAA (SEQ ID NO: 11), GCAAGGCTGAGGTTCACAGG (SEQ ID NO: 12), GTGTTACCGAATGGCATGG (SEQ ID NO: 13), TTACCGAATGGCATGGTGG (SEQ ID NO: 14), GGGAAGATTGAAGATTAGC (SEQ ID NO: 15), GGTGTTACCGAATGGCATGG (SEQ ID NO: 16), TACACCGCCACCATGCCATT (SEQ ID NO: 17), GCGGTGTAGGAGACCTGAT (SEQ ID NO: 18), a sequence with at least 65% sequence identity with any one of SEQ ID NOS: 10-18, a sequence with at least 70% sequence identity with any one of SEQ ID NOS: 10-18, a sequence with at least 75% sequence identity with any one of SEQ ID NOS: 10-18, a sequence with at least 80% sequence identity with any one of SEQ ID NOS: 10-18, a sequence with at least 85% sequence identity with any one of SEQ ID NOS: 10-18, a sequence with at least 90% sequence identity with any one of SEQ ID NOS: 10-18, a sequence with at least 95% sequence identity with any one of SEQ ID NOS: 10-18, a sequence with at least 99% sequence identity with any one of SEQ ID NOS: 10-18, or combinations thereof.

In some embodiments, the guide RNAs of the CRISPR system direct the nuclease to a region of MB. In some of such embodiments, such guide RNAs direct the nuclease to an MB region that includes, without limitation, GCCAGACTAGCATCTGGGA (SEQ ID NO: 1), TCCTCTTTAGAAGCCACCA (SEQ ID NO: 2), GGAAGGTATAAAAGCCCTTC (SEQ ID NO: 3), TCTTCTTCAGACTGCGCCA (SEQ ID NO: 4), AGAGCAAGTATGGGCTCACT (SEQ ID NO: 5), TCTTCTTCAGACTGCGCCAT (SEQ ID NO: 6), CTTCTTCAGACTGCGCCATG (SEQ ID NO: 7), GGTCTTCTTCAGACTGCGCCA (SEQ ID NO: 8), AAGGTATAAAAACGCCCTT (SEQ ID NO: 9), a sequence with at least 65% sequence identity with any one of SEQ ID NOS: 1-9, a sequence with at least 70% sequence identity with any one of SEQ ID NOS: 1-9, a sequence with at least 75% sequence identity with any one of SEQ ID NOS: 1-9, a sequence with at least 80% sequence identity with any one of SEQ ID NOS: 1-9, a sequence with at least 85% sequence identity with any one of SEQ ID NOS: 1-9, a sequence with at least 90% sequence identity with any one of SEQ ID NOS: 1-9, a sequence with at least 95% sequence identity with any one of SEQ ID NOS: 1-9, a sequence with at least 99% sequence identity with any one of SEQ ID NOS: 1-9, or combinations thereof.

### Nucleic Acid Sequences

The methods of the present disclosure may insert various nucleic acid sequences at a region of a gene. Additionally, the systems of the present disclosure may include various nucleic acid sequences for insertion at a region of a gene. Moreover, the modified cells of the present disclosure may include various inserted nucleic acid sequences. For instance, in some embodiments, the nucleic acid sequence includes a gene. In some embodiments, the nucleic acid sequence expresses a therapeutic protein. In some embodiments, the therapeutic protein includes human factor 9 (hF9), human factor 8 (hF8), dystrophin, microdystrophin (DMD), and other replacements for circulating factors and neuromuscular disease.

The nucleic acid sequences of the present disclosure may be in various forms. For instance, in some embodiments, the nucleic acid sequence lacks a promoter. In some embodiments, the nucleic acid sequence is inserted in the form of a double stranded DNA. In some embodiments, the nucleic acid sequence is inserted in the form of a single stranded DNA. In some embodiments, the nucleic acid sequence is inserted as part of a plasmid.

### Expression of Nucleic Acid Sequences in Cells

The nucleic acid sequences of the present disclosure may be expressed in various cells by the methods and systems of the present disclosure. Additionally, the modified cells of the present disclosure can include various types of cells. For instance, in some embodiments, the cells include, without limitation, a skeletal muscle cell, a human cell, a mouse cell, a diseased cell, or combinations thereof. In some embodiments, the cell includes a human cell. In some embodiments, the cell includes a skeletal muscle cell. In some embodiments, the skeletal muscle cell includes a myoblast. In some embodiments, the skeletal muscle cell includes a myotube.

The inserted nucleic acid sequences of the present disclosure may be expressed in cells in various manners. For instance, in some embodiments, the insertion of a nucleic acid sequence at a region of a gene results in stable expression of the nucleic acid sequence in a cell through an endogenous promoter of the gene. In some embodiments, the insertion of a nucleic acid sequence at a region of a gene results in stable expression of the nucleic acid sequence in a cell through an endogenous promoter of CKM. In some embodiments, the insertion of a nucleic acid sequence at a region of a gene results in stable expression of the nucleic acid sequence in a cell through an endogenous promoter of MB.

### Modes and Applications of Nucleic Acid Sequence Expression

The nucleic acid sequences of the present disclosure may be inserted into gene regions in various modes and for various applications. For instance, in some embodiments, nucleic acid sequence insertion occurs in vitro. In some embodiments, the in vitro insertion occurs by a method that includes, without limitation, transfection, transduction, viral mediated transduction, lipid nanoparticle transfection, or combinations thereof.

The in vitro insertion of nucleic acid sequences into cells can have numerous applications. For instance, in some embodiments, the methods and systems of the present disclosure may be utilized for in vitro expression of proteins. In some embodiments, the in vitro modified cells and the modified cells of the present disclosure could be utilized for numerous applications, such as cell therapies (e.g., muscle-directed cell therapies).

In some embodiments, nucleic acid sequence insertion occurs in vivo in a subject. In some embodiments, the in vivo insertion occurs by administering the nucleic acid sequences and/or systems of the present disclosure to the subject.

The nucleic acid sequences and systems of the present disclosure may be administered to various subjects. For instance, in some embodiments, the subject is a human being. In some embodiments, the subject is a non-human mammal. In some embodiments, the non-human mammal includes, without limitation, a horse, a rabbit, a mouse, a rat, a pig, a sheep, a cow, a dog, or a cat. In some embodiments, the non-human mammal is a domestic animal, such as a dog or a cat.

The nucleic acid sequences and systems of the present disclosure may be administered to subjects in various manners. For instance, in some embodiments, the administration occurs by a method that includes, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, or combinations thereof.

The in vivo insertion of nucleic acid sequences into cells can have numerous applications. For instance, in some embodiments, the expressed nucleic acid sequence is utilized to treat or prevent a disease in a subject. In some embodiments, the expressed nucleic acid sequence may be utilized for correction of a disease in a subject. In some embodiments, the expressed nucleic acid sequence may be utilized for tissue repair or regeneration in a subject. In some embodiments, the expressed nucleic acid may be utilized for reversal of age related decline.

The methods, systems and modified cells of the present disclosure may be utilized to treat, prevent or correct various diseases. For instance, in some embodiments, the disease is a genetic disease or disorder. In some embodiments, the disease is a muscle disease. In some embodiments, the disease is a genetic disorder with spectrum mutations, such as hemophilia A, hemophilia B, lyosomsomal storage disorders, neuromuscular disorders, muscular dystrophies, and muscular atrophies.

### Applications and Advantages

The methods, systems and modified cells of the present disclosure can have numerous applications and advantages. For instance, in some embodiments, the methods, systems and modified cells of the present disclosure may be utilized to improve gene therapy for genetic diseases. In some embodiments, the methods, systems, and modified cells of the present disclosure may be integrated into existing gene therapy solutions to improve their safety, efficacy, and sustainability. In some embodiments, such applications can enhance the treatment outcomes for patients with genetic discases.

In some embodiments, the methods, systems, and modified cells of the present disclosure can also be used to develop new gene therapy solutions for a broad range of genetic diseases. For instance, in some embodiments, the methods, systems and modified cells of the present disclosure can be utilized to design more efficient and targeted gene therapies with better safety and efficacy profiles.

In some embodiments, the methods, systems, and modified cells of the present disclosure can also be used to enhance precision medicine. For instance, in some embodiments, the methods, systems, and modified cells of the present disclosure can enhance precision medicine by enabling targeted gene therapies for specific patient populations with genetic diseases. This can help personalize treatment approaches based on the patient's genetic makeup, resulting in better treatment outcomes.

In some embodiments, the methods, systems, and modified cells of the present disclosure can be used to advance regenerative medicine. For instance, in some embodiments, the methods, systems, and modified cells of the present disclosure can be used to advance regenerative medicine approaches by enabling the efficient and sustainable expression of genes that promote tissue repair and regeneration. This can potentially improve the treatment outcomes for patients with tissue injuries or degenerative diseases.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Precision and Efficacy of RNA-guided DNA Integration in High-Expressing Muscle Loci

Gene replacement therapies primarily rely on adeno-associated viral (AAV) vectors for transgene expression. However, episomal expression can decline over time due to vector loss or epigenetic silencing. CRISPR-based integration methods offer promise for long-term transgene insertion. While the development of transgene integration methods has made substantial progress, identifying optimal insertion loci remains challenging. Skeletal muscle is a promising tissue for gene replacement owing to low invasiveness of intramuscular injections, relative proportion of body mass, the multinucleated nature of muscle, and the potential for reduced adverse effects.

Leveraging endogenous promoters in skeletal muscle, Applicant evaluated in this Example two high-expressing loci using homology-independent targeted integration (HITI) to integrate reporter or therapeutic genes in mouse myoblasts. Applicant hijacked the muscle creatine kinase (Ckm) and myoglobin (Mb) promoters by co-delivering CRISPR-Cas9 and a donor plasmid with promoter-less constructs encoding green fluorescent protein (GFP) or human Factor IX (hFIX). Additionally, Applicant deeply profiled genome and transcriptome outcomes from targeted integration and evaluated the safety of the proposed sites. As such, this Example introduces a proof-of-concept technology for achieving high-level therapeutic gene expression in skeletal muscle, with potential applications in targeted integration-based medicine and synthetic biology.

In this Example, Applicant identified new potential safe-harbor sites in skeletal muscle that offer secure and stable integration, facilitating gene replacement. Choosing skeletal muscle as a gene therapy integration site can reduce procedure invasiveness and complexity. Its unique syncytial nature is less likely to create negative effects in vivo, with minimal local or systemic adverse effects associated with intramuscular injection were reported in a systematic review for AAV gene therapies. Owing to its local delivery, the exposure to circulating neutralizing antibodies, such as AAV pre-existing antibodies against the viral capsid is minimized, ensuring efficient tissue transduction. Skeletal muscle's well-vascularized nature allows secreted proteins to enter circulation and intramuscular administration of AAV vector that lead to the secretion of functional proteins has been previously demonstrated.

In this Example, Applicant devised a strategy to leverage endogenous promoters in muscle cells for expressing either the reporter gene or a therapeutic gene. Applicant adapted the currently established safe-harbor characterization to evaluate two high-expressing skeletal-muscle loci. Applicant applied homology-independent targeted integration (HITI) to mediate transgene integration, introducing a reporter gene or therapeutic gene (human factor 9, hF9) to express human factor 9 protein (hFIX) from the identified loci. By delivering a promoter-less transgene, Applicant was able to detect both protein expression in the reporter gene and hFIX. Applicant deeply profiled genome and transcriptome outcomes after gene-editing outcomes using high-throughput sequencing, unidirectional sequencing, and nanopore long-read sequencing. Overall, the data support RNA-guided DNA-integration strategies as effective therapies for restoring desired gene expression in muscle and can be extended to synthetic biology applications.

### Example 1.1. Selected Muscle-Specific Integration Sites Have High Expression and a Predicted Favorable Safety Profile

The selection of integration sites involves combining established criteria for potential safe-harbor sites with insights from prior in vivo studies that employed the highly expressing locus, albumin (ALB), to express therapeutic genes in the liver. Applicant has tailored this strategy to target integration sites within skeletal muscles. To pinpoint suitable sites, Applicant referred to a comprehensive analysis of public transcriptomics studies on skeletal muscle in humans and mice. A. M. Abdelmoez et al., American Journal of Physiology-Cell Physiology 318, C615-C626 (2020). Through their thorough examination of the compilation and the application of rigorous quality control, normalization, and annotation using official human gene names, Applicant identified the top 10 genes specifically expressed in skeletal muscle and demonstrated high expression levels in both humans and mice (Table 1). The selection of these genes is rooted in the goal of ensuring robust and consistent muscle-specific promoter expression in different models of organisms. Next, Applicant evaluated the essentiality of each gene and the safety implications associated with hijacking its endogenous promoter.

TABLE 1
A comparison of highly expressed genes in skeletal muscle
tissues between mice and humans. The data were extracted
from A. M. Abdelmoez et al., American Journal of Physiology-
Cell Physiology 318, C615-C626 (2020), which compiled
skeletal muscle tissue gene expressions from the Gene
Expression Omnibus public database.

| | |
|---|---|
| Human | Mouse |

| | Gene | RPKM | SE | RPKM | SE |
|---|---|---|---|---|---|
| 1 | ACTA1 | 7.03 | 0.27 | 7.14 | 0.14 |
| 2 | CKM | 6.91 | 0.24 | 5.65 | 0.60 |
| 3 | MB | 6.63 | 0.46 | 6.14 | 0.49 |
| 4 | MYH7 | 6.72 | 0.33 | 2.26 | 2.80 |
| 5 | MYL2 | 6.87 | 0.26 | 3.35 | 2.52 |
| 6 | DES | 6.11 | 0.44 | 3.35 | 1.68 |
| 7 | TNNC2 | 6.28 | 0.54 | 6.93 | 0.46 |
| 8 | TNNI | 6.31 | 0.43 | 1.93 | 2.73 |
| 9 | MYL3 | 5.79 | 0.24 | 3.46 | 2.23 |
| 10 | TTN | 6.11 | 0.70 | 0.63 | 4.52 |

RPKM: Reads Per Kilobase per Million mapped reads, SE: standard error.

Skeletal actin (ACTA1) is on top of the list as the highest expressed gene in skeletal muscle. However, skeletal actin is the main actin isoform in skeletal muscle and it plays an essential role, alongside myosin, in facilitating muscle contraction. Further, ACTA1 disruption in newborn mice causes early demise. In light of this, Applicant propose creatine kinase (CKM) and myoglobin (MB) as genomic integration sites for skeletal muscle.

While Applicant's approach does not involve knocking out the Ckm or Mb gene, these selections are based on the viability and state of skeletal muscle after a knock-out experiment. Mice lacking Ckm are viable and exhibit no changes in absolute muscle force; but, they show an inability to engage in burst activity. Mice without myoglobin are also viable with no distinct phenotype apart from depigmentation of the cardiac muscle and cardiac adaptations regarding oxygen delivery.

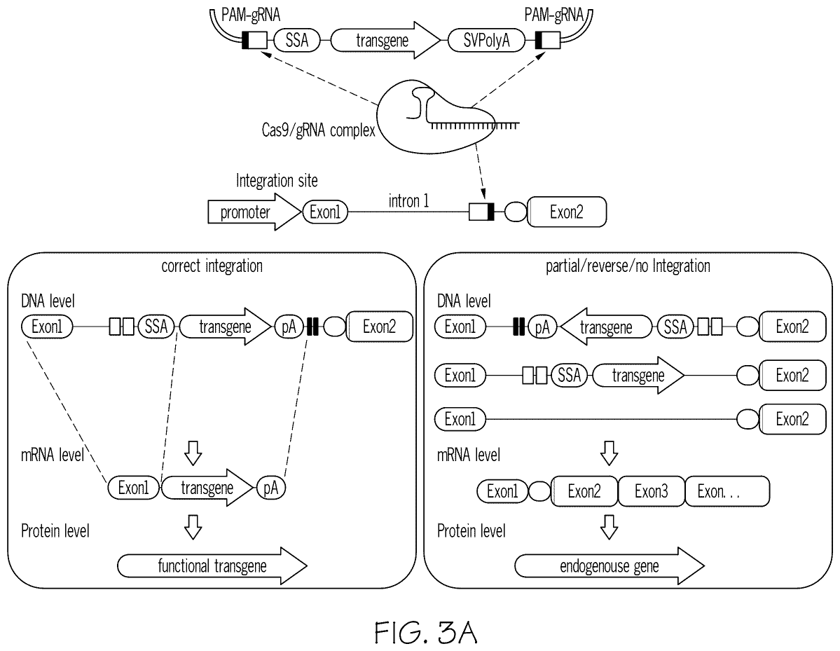

To identify if these sites could serve as potential genomic safe-harbor sites, Applicant assessed the suitability of creatine kinase and myoglobin as safe-harbor sites in comparison to currently known safe-harbor sites. Scores were assigned based on previously established and widely accepted criteria (FIG. 2; Table 2).

TABLE 2
A display of the assessment of currently widely used safe-
harbor sites (AAVS1, CCR5, and hROSA26) and proposed safe-
harbor sites. The assessment was performed based on the ideal
criteria of safe-harbor sites. Each criterion was evaluated
in the UCSC genome browser track following recommendations.

| | Criteria (FIG. 2) | | | | | | | |
|---|---|---|---|---|---|---|---|---|
| Location | 1 | 2 | 3 | 4 | 5 | 6 | 7 | 8 |
| AAVS1 | No | Yes | No | Yes | Yes | No | Yes | Yes |
| CCR5 | No | Yes | No | Yes | Yes | No | Yes | Yes |
| hROSA26 | No | Yes | No | Yes | Yes | No | Yes | Yes |
| CKM intron | No | Yes | No | Yes | Yes | No | Yes | Yes |
| MB intron | Yes | Yes | No | Yes | Yes | No | Yes | Yes |

Table 2 indicates that Applicant's proposed sites did not meet all the criteria. However, even the most commonly used safe-harbor sites fail to achieve a 100% clearance based on these criteria. Interestingly, MB shows better results in a free oncogene region criterion compared to CKM and other sites.

### Example 1.2. Targeted Integration Into Ckm and Mb Lead to Expression of a Promoter-Less Reporter

To experimentally assess transgene expression from proposed sites (intron 1 of CKM or MB), Applicant performed a targeted integration approach to knock in a gene construct encoding a promoter-less green fluorescence reporter protein (GFP) into mouse myoablsts (Ckm or Mb). Applicant identified the knock-in site within the intron preceding the coding sequence of the gene, ensuring that the non-edited or inaccurately edited copy maintains functional gene expression via mRNA splicing. This approach also provides flexibility in transgene selection, enabling the incorporation of specific features, such as a signaling peptide or a transgene that does not require a signaling peptide.

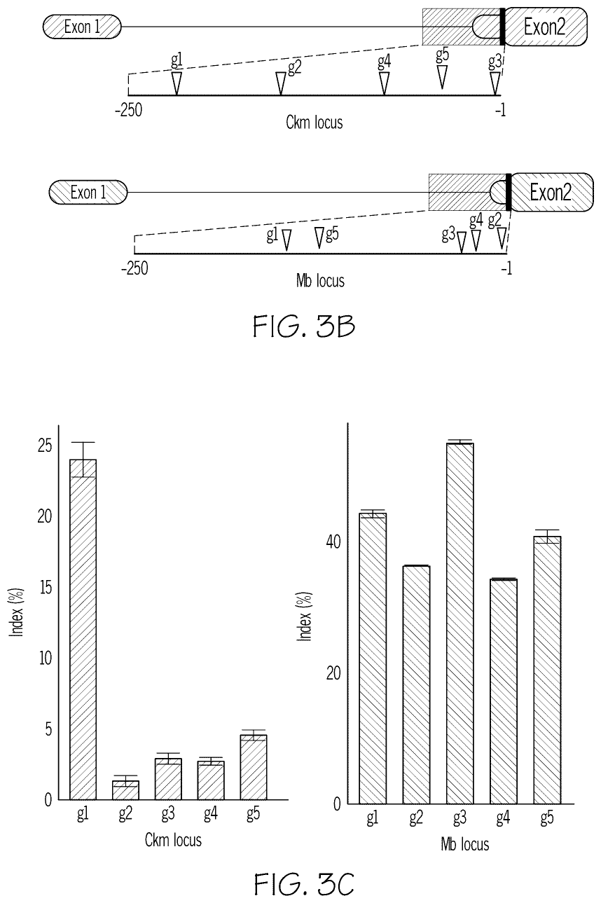

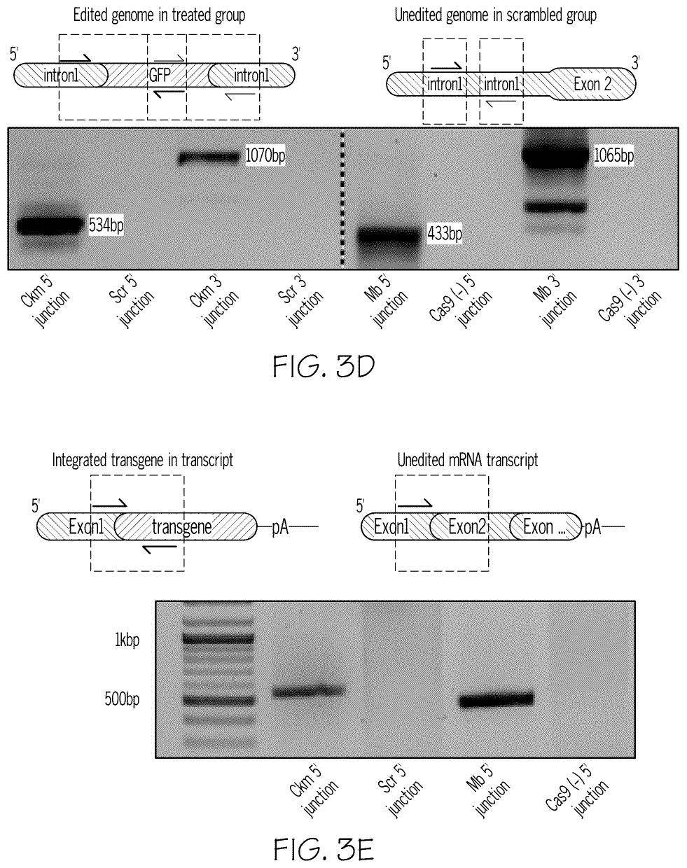

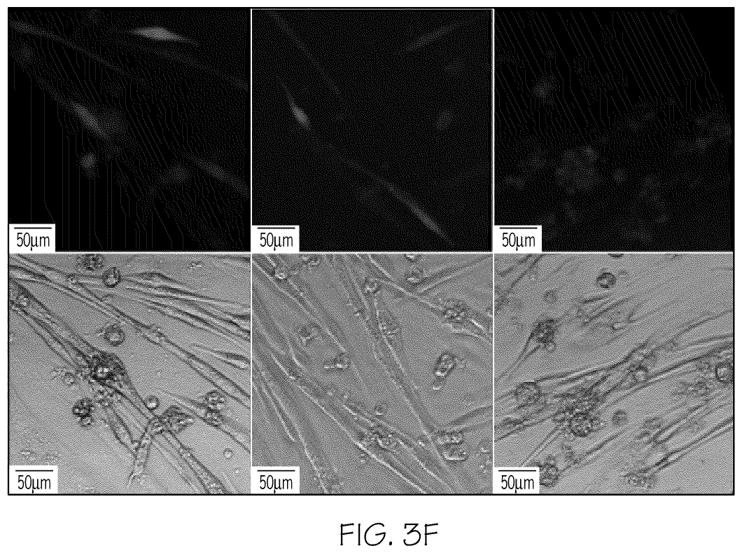

To integrate GFP, Applicant used a CRISPR/Cas9-based genome editing strategy that uses the HITI method capitalizing the NHEJ pathway that is accessible in muscle cells (FIG. 3A). This approach designs Cas9 target sites in the donor DNA as reverse complements of the genomic target site to facilitate the re-cleavage of reverse-integration products, providing a means to dictate the specific directionality of the knock-in. Applicant applied this strategy to C2C12 immortalized mouse myoblast cell lines as it is the most commonly used cellular model to study murine skeletal muscle in vitro.

As a proof of concept, Applicant identified five guide RNA (gRNA) sites within intron 1 of each gene, positioned distal to the enhancer and proximal to exon 2, specifically within the last 250 base pairs leading up to the noncoding portion of exon 2 (FIG. 3B) based on CRISPOR web tool and screened their cutting efficiency in NIH3T3 cell line. Additionally, Applicant conducted PCR-enriched next-generation sequencing (NGS) using Iseq short-read sequencing to quantify the efficiency of gRNA in the C2C12 cell line. Within the Ckm gene, g1 exhibited the highest efficiency, making it the selected gRNA for HITI-mediated GFP integration experiments. In the Mb gene, although g3 demonstrated slightly highest cutting efficiency, it targeted the non-coding exon sequence. Therefore, Applicant opted for g1 as the gRNA for the downstream integration experiment (FIGS. 3B and 3C).

Applicant used Lipofectamine 3000 to co-transfect C2C12 muscle myoblasts with a ratio of 2:2:1 for SpCas9, guide RNA, and site-specific promoter-less insert plasmids, respectively. Three days after transfection, Applicant performed a genotyping PCR to confirm the integration. PCR primers were used spanning the intron of Ckm or Mb to the 5′ junction of the GFP insert, and from the 3′ junction of the GFP insert to the intron of Ckm or Mb. Applicant detected bands at the expected length of chimeric Ckm-GFP or Mb-GFP integration in both 5′ and 3′ end junctions at the genomic DNA and cDNA levels, but no bands were observed in the control groups (FIGS. 3D and 3E). In myotubes, after differentiation, Applicant observed GFP expression in the CRISPR-treated group in both loci, while the scrambled group did not exhibit GFP expression. This suggests that the GFP expression was driven by the Ckm or Mb promoter (FIG. 3F).

### Example 1.3. Targeted Integration of a Promoter-Less Human F9 Gene at Ckm and Mb Leads to Sustained Expression of hF9

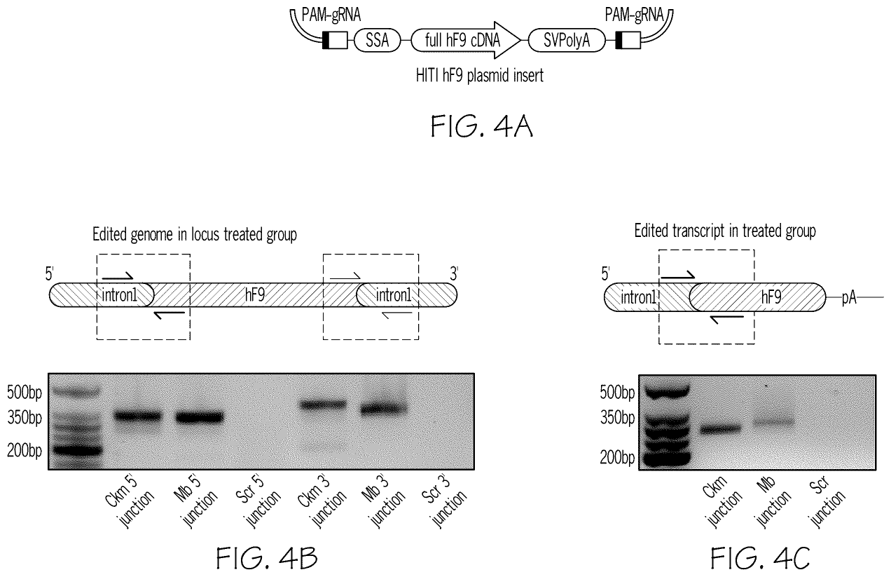

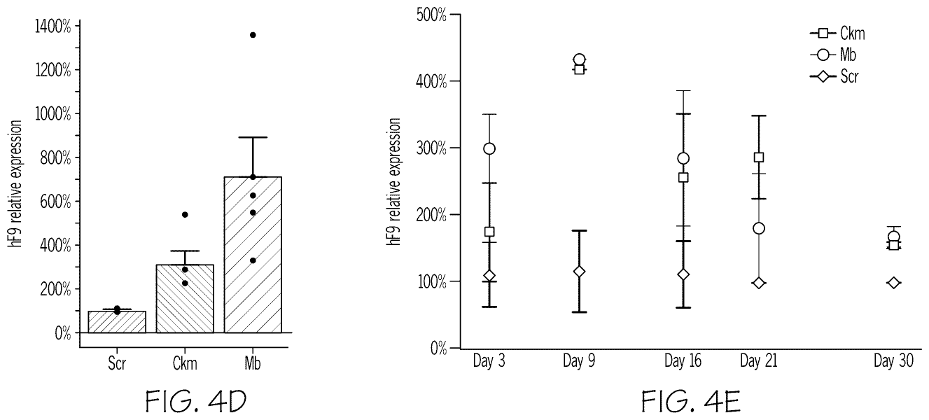

Next, Applicant sought to investigate whether this strategy applies to therapeutic genes. The GFP transgene in the HITI insert plasmid was substituted with a transgene encoding human factor IX (hF9) (FIG. 4A). The selection of hF9 as a proof of concept for therapeutic gene integration was motivated by previous reports indicating that even with <1% of targeted integration events of hF9 under the albumin promoter, it proved adequate to attain 5-20% of FIX levels, effectively correcting bleeding in hemophilia B mice. In this approach, Applicant utilized the complete coding sequence of hF9, given the absence of signaling peptides in the Ckm or Mb gene.

Following a method similar to GFP transfection, Applicant co-delivered a site-specific hF9 HITI insert plasmid with SpCas9 and Ckm or Mb gRNA into C2C12 myoblasts using Lipofectamine 3000. Post-transfection, as observed in GFP integration, Applicant verified hF9 integration exclusively within the CRISPR-mediated HITI group at both genomic DNA and cDNA levels in both sites (FIGS. 4B and 4C).

To evaluate changes in gene expression between groups, Applicant conducted qRT-PCR using FAM probe primers spanning from exon 6 to exon 7 of hF9. The Ppia gene served as a housekeeping gene for normalizing expression levels. This selection was based on Applicant's data that showed the Ppia gene has the most consistent expression in both myoblasts and myotubes. In contrast, Gapdh has variable expression levels between the myoblast and myotube stages. Furthermore, Applicant evaluated four different reference genes from a previous study to identify the most consistent expression in both myoblast and myotubes.

During the myotube stage, the relative expression of hF9 was found to be elevated in the CRISPR-mediated HITI group compared to the scrambled group, reaching up to a 3-fold increase in Ckm-mediated expression and up to a 10-fold increase in Mb-mediated expression (FIG. 4D). Additionally, to assess if this increased mRNA expression translates to hFIX protein expression, Applicant performed an hFIX sandwich ELISA assay on cell media after myotube differentiation. Applicant observed an increased level of protein expression in both Ckm and Mb-treated cell serum relative to a scrambled control.

Furthermore, to determine whether identified sites supporting long-term stable hF9 expression, Applicant maintained the myoblast culture for 30 days. Despite Applicant's efforts to incorporate a reporter gene marker in Cas9 for cell sorting, Applicant encountered difficulties in maintaining the sorted cultured cells. This obstacle has also been noted in previously published literature, which indicates that myoblasts tend to lose their differentiation potential after single-cell cloning, and those that are successfully edited often fail to survive the stress of sorting. Since generating clonal populations was not feasible, Applicant opted to gather informative data from bulk populations by routinely passaging the cells when they reached 60-70% confluency, before spontaneously differentiating to myotubes.

Following the 30-day culture, Applicant detected integration in DNA level. In the time-course analysis, hF9 gene expression was assessed at various intervals starting from 3 days post-transfection, with cell passaging every two to three days, followed by differentiation from day 23 to 30. Despite the continuous passaging of cells, this longitudinal study demonstrated generally consistent expression of hF9 throughout the observation period. The variance in fold change observed between fully differentiated myotubes in FIGS. 4D-4E could be attributed to the continuous passaging process, which involved regular splitting of the cells.

### Example 1.4. Multi-Pronged Targeted Next-Generation Sequencing and Long-Read Sequencing Show Relative Precision of HITI-Based Integration

While Applicant has confirmed that the Ckm and Mb endogenous promoter can express the promoter-less protein, the precision of editing was unknown. Imprecise integration could negatively impact protein expression. As such, Applicant evaluated factors affecting protein expression such as the precision and the efficiency of integration. Applicant performed PCR-enriched short-read sequencing for fragments spanning the genomic insertion site or chimeric transcript to evaluate if the integrated chimeric sequence is as precise based on the mechanism of HITI or if unintentional on-target events could be detected.

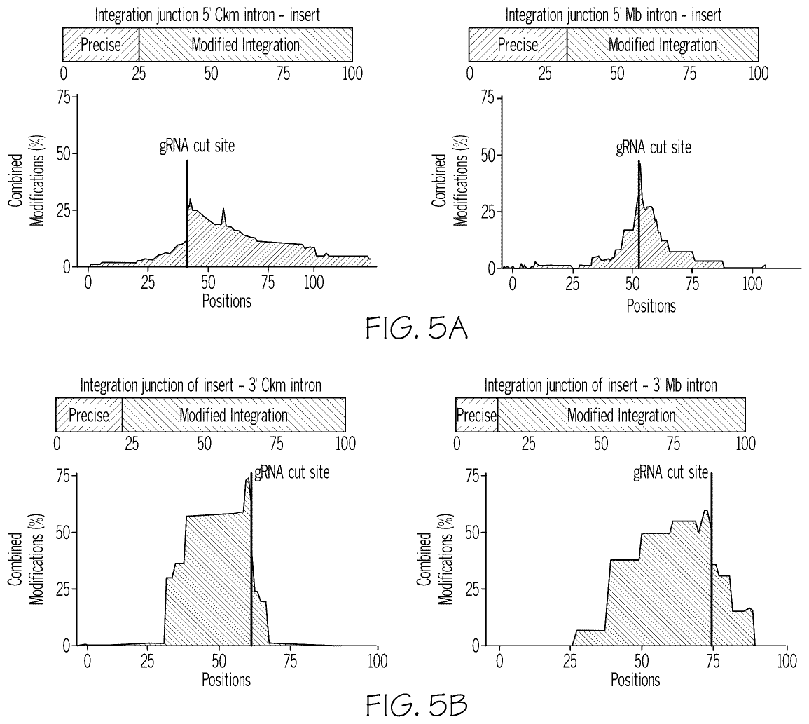

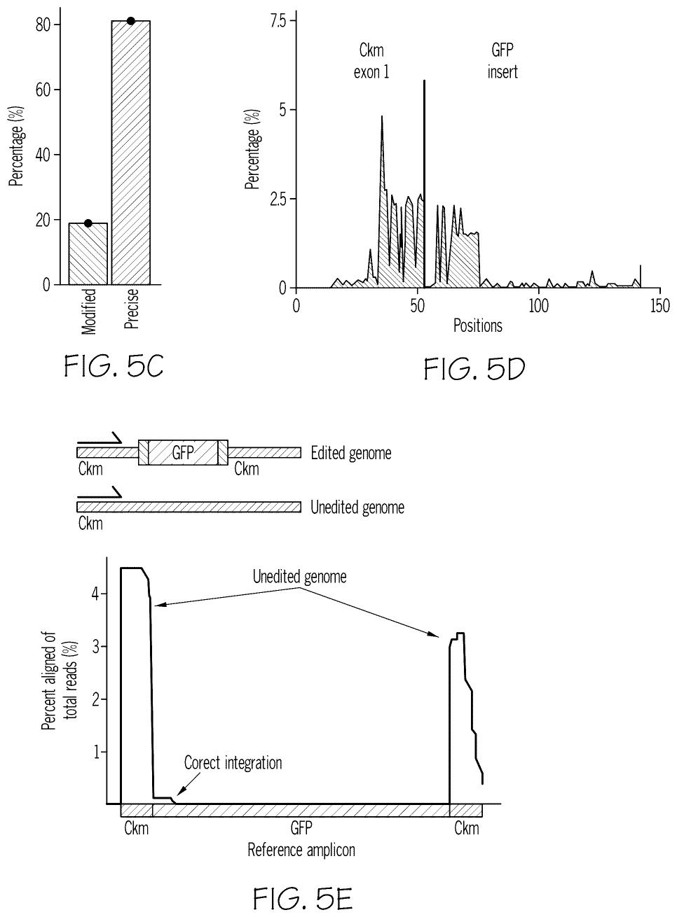

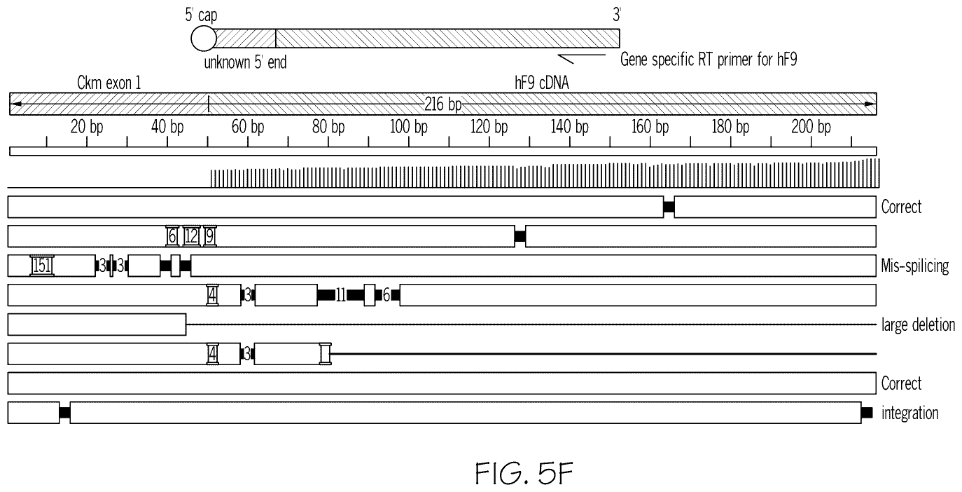

Genomic DNA-level analysis revealed levels of precise integration ranging from 15% to 35% in 5′ and 3′ integration junctions in both loci (FIGS. 5A-5B). The modified sequence predominantly resulted from NHEJ repair at gRNA cut sites with preferential removal of sequence on the inserted sequence (FIGS. 5A-5B). Although genomic DNA-level precision was not optimal, at the mRNA level, Applicant saw a higher average of precision integration around 80% of precise integration (FIG. 5C) with some modifications on the junction site of exon 1 Ckm and start codon GFP (FIG. 5D).

Precision integration data provide a selective perspective on how effectively this method incorporates the transgene insert. However, selection of primers and PCR will bias the result. Moreover, this method does not quantify the proportion of integrated sequences due to the PCR-enrichment method that selectively amplifies the integrated sequences. To quantify the integration efficiency, Applicant conducted a Uni-Directional Targeted Sequencing methodology (UDiTaS) that is based on Tn5 transposase-assisted tagmentation short-read sequencing. This method incorporates a unique molecular identifier (UMI) to remove PCR amplification bias. Applicant quantified the number of sequences integrated with their transgene by tagging primers specific to the Ckm intron before the gRNA cut site (FIG. 5E). Reading from this site will capture unedited, precisely edited, and unintentionally modified alleles.

In FIG. 5E, Applicant deduplicated the UMI and aligned the resulting bam files to the expected fusion of Ckm intron-GFP integration. After implementing filters to verify that the plotted reads were authentic and not sequencing artifacts, the analysis revealed that correct GFP integration represented 2.8% of the total aligned reads. This percentage was calculated by dividing the proportion of edited reads by the proportion of unedited reads that aligned with the reference amplicon from the total reads (4%). Separately, a primer specific to the GFP insert genome was used in conjunction with the same transposon-specific primer to map genome-wide GFP episome integration into the mouse genome. Following similar pipeline with UdiTaS analysis, Applicant did not detect GFP integration in other locations in the genome.

The cDNA selective PCR-enriched sequencing method revealed a high percentage of precise integration. However, it may overlook large structural variants. Therefore, to get a more comprehensive picture of the precision, Applicant aimed to characterize the 5′ integration junction of the fusion between Ckm and the transgene at the transcriptome level. This approach is motivated by the expectation that intron rearrangements would be spliced out in mRNA processing. To achieve this, Applicant conducted 5′ rapid amplification of cDNA ends (RACE) using cDNA from Lipofectamine-transfected cells and amplified Ckm-hF9 fusion transcripts using a gene-specific primer (GSP) positioned at the end of the hF9 region. Long-read nanopore sequencing confirmed the addition of exon 1 of Ckm in the 5′ region of hF9. However, Applicant also observed several structural rearrangement events, including significant deletions and insertions (FIG. 5F). Each line in the figure represents a single alignment. Upon investigation of the 151 bp insertion, Applicant determined that it originated from the splice acceptor region incorporated from the HITI insert plasmid. This suggests the occurrence of mis-splicing events at the transcriptome level.

### Example 1.5. RNA Sequencing Reveals Differentially Expressed Genes After Targeted Integration

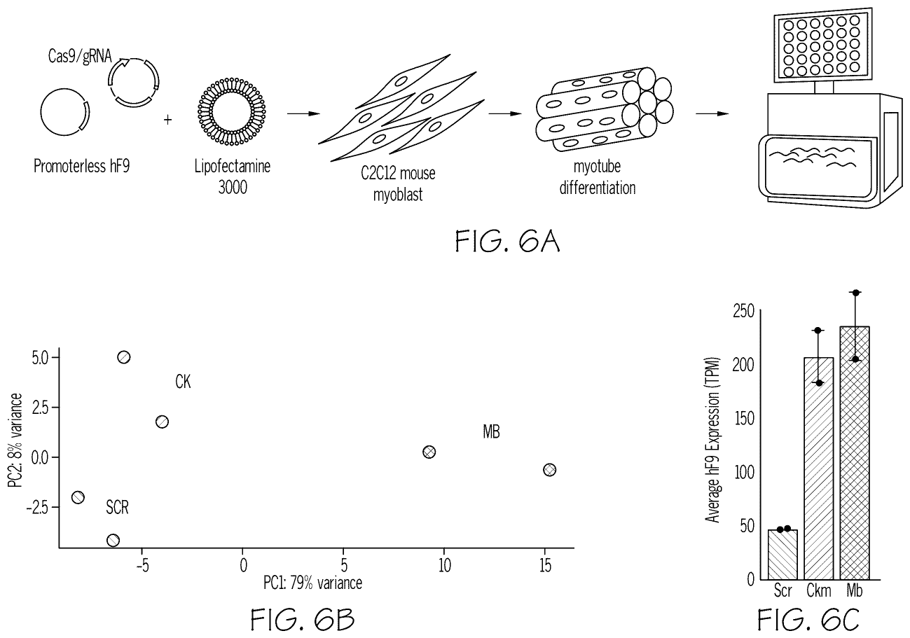

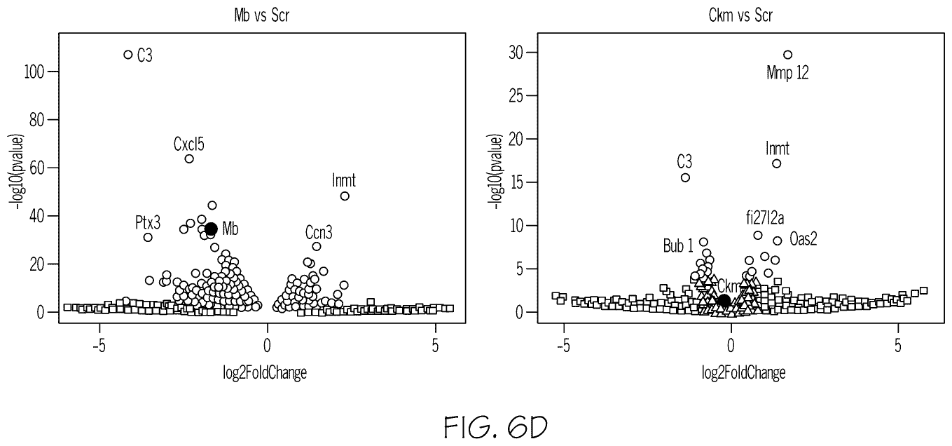

To investigate whether the hF9 targeted integration into identified sites resulted in alterations in the overall transcriptome profiles, bulk RNA-seq analysis was performed. After seven days of myotube differentiation, samples exhibiting the high hF9 expression levels in Ckm and Mb based on qPCR analysis were compared with scrambled-treated cells from the same experimental batch (FIG. 6A). Paired-end sequencing on the DNBSEQTM Sequencing System from BGI with an average read length of 100 bp was used for sequencing two biological replicates from each treatment. Principal component analysis (PCA) was performed, followed by visualization of each sample in two dimensions using the first two principal components (PCs). PCA revealed biological variation between samples in the same treatment groups. However, Applicant observed transcriptional similarity within the Ckm-integrated and scrambled group and transcriptional variations in Mb-treated samples compared to the scrambled group (FIG. 6B).

To assess human F9 expression in the samples, Applicant normalized the transcript expression level (TPM) and found that hF9 is highly expressed in both Ckm and Mb loci compared to the scrambled group (FIG. 6C). Subsequent differential gene expression analysis was performed to evaluate changes in the transcriptome post-treatment. The analysis revealed several genes that are both downregulated and upregulated in both Ckm and Mb-treated samples, such as C3 and Inmt genes. Differentially expressed genes were observed to be dispersed across various chromosomes (e.g., C3 on chromosome 17, Inmt on chromosome 6, and Mmp12 on chromosome 12), rather than being clustered within the targeted chromosome (Ckm on chromosome 7; Mb on chromosome 15), where more local contacts are expected to occur. Consistent with the PCA results, more genes were differentially expressed in Mb-treated samples compared to Ckm-treated samples (FIG. 6D). Additionally, downregulation of the Mb gene was observed upon editing.

Applicant's current work aims to address several significant challenges associated with AAV vector-based in vivo gene therapy, including long-term efficacy, promoter or transgene silencing, and the safety of transgene integration. To enhance the sustainability of the treatment, targeted integration into the host genome has emerged as a potential solution. However, the location of where the integration must be strategic since the integrating vector with the constitutive promoter might pose risks. Despite their ability to facilitate robust and consistent transgene expression, such promoters are associated with increased risks, including an elevated likelihood of inactivation, amplified toxicity resulting from transgene overexpression, off-target transgene expression, and the potential for severe immune responses due to inadvertent transgene expression in antigen-presenting cells.

Utilizing endogenous promoters for transgene expression can circumvent potential silencing mechanisms associated with exogenous promoters. In this Example, Applicant identified two highly expressed genes, Ckm and Mb, in skeletal muscle and exploited the transcriptional output of their endogenous promoters. Applicant's findings demonstrate that these loci can drive the expression of promoter-less GFP and enhance RNA expression of promoter-less hF9 in C2C12 cells upon plasmid transfection using lipofection. However, due to the limited efficiency of lipofection in myoblasts, compounded by the generally low efficiency of integration, Applicant confirmed that the efficiency of transgene integration at the genomic level is approximately 3% through the UDiTaS sequencing approach. Despite this modest level of integration, Applicant observed the expression of promoter-less GFP and increased RNA expression of hF9 compared to a control, representing the episomal vector without Cas9.

Furthermore, it is noteworthy that the expression levels of Ckm and Mb in C2C12 cells were not as high as reported in skeletal muscle tissue. This disparity may contribute to the lower expression of GFP and hFIX protein observed. Additionally, regarding the hFIX protein results, the rich presence of collagen IV in skeletal muscle may facilitate the local attachment of FIX, limiting its release into circulation. Previous studies exploring the production of ectopic FIX protein in muscle have suggested the potential use of hFIX variants harboring mutations such as lysine to alanine at residue 5 (K5A) or valine to lysine at residue 10 (V10K), which exhibit reduced binding to endothelial cells while maintaining normal clotting activity, thereby enabling synthesis of hFIX in skeletal muscle. Hence, investigating the expression of this variant under a muscle endogenous promoter in muscle cells could potentially result in increased hFIX release into circulation.

The general safety evaluation of the proposed sites indicates that the group treated with Mb exhibits alterations in the global transcriptome. The downregulation of the Mb gene may be attributed to the higher indel activity of the CRISPR/gRNA complex at that target site compared to the indel activity at the Ckm target sites. Despite observing minimal changes in the expression of upregulated and downregulated genes at the Ckm locus, which may suggest passing initial safety validation, a more comprehensive analysis is still warranted. C3 downregulated expression was reported in the acute stage of infection suggesting that C3 participates in inflammation and slight upregulation of Inmt was reported in skeletal muscle under spaceflight conditions.

Applicant's sequencing studies also show patterns of deletions following integration induced by double-strand breaks (DSBs). These sequences demonstrate that indel-induced damage tends to occur toward the insertion site, as evidenced by the selected PCR-enriched integration sequencing. This trend is consistently observed across loci and in both 5′ and 3′ integrations. This insight is valuable for future template design efforts, emphasizing the importance of incorporating padding sequences before the coding sequence of the transgene to mitigate chewback into the desired gene before integration can occur.

Applicant's approach of integrating the transgene into intronic regions offers the advantage of splicing, as indels occurring at the intronic level are typically spliced out, resulting in more precise mRNA. Despite selecting a robust splice acceptor from a highly conserved gene in skeletal muscle, based on Applicant's 5′ RACE results, Applicant observed instances of missplicing due to the failure of the splice acceptor to be properly excised. To date, there is a lack of practical guidelines available to determine the ideal length of splice acceptors or splice donors for a given gene. Thus, design of the splice acceptor allows additional optimization of gene expression.

In summary, this Example presents a proof-of-concept technology for achieving high-level therapeutic gene expression in skeletal muscle. Such methods hold promise for advancing targeted integration-based medicine or synthetic biology approaches, effectively transforming skeletal muscle into a biofactory capable of producing desired therapeutic agents.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A method of expressing a nucleic acid sequence in a cell, said method comprising:
inserting the nucleic acid sequence at a region of a gene,
wherein the gene comprises a creatine kinase (CKM) gene, a myoglobin (MB) gene, or combinations thereof, and
wherein the inserted nucleic acid sequence becomes expressed in the cell by a promoter of the gene.

2. The method of claim 1, wherein the insertion occurs by a method selected from the group consisting of site-specific nucleic acid integration, site-specific recombination, RNA-guided nucleic acid integration, insertion by a gene editing system, insertion by a clustered regularly interspaced short palindromic repeats (CRISPR)/nuclease system (CRISPR system), insertion by a non-DSB (double strand break) CRISPR system with integrase or transposase combination, homology-independent targeted integration (HITI), or combinations thereof.

3. The method of claim 1, wherein the insertion of the nucleic acid sequence comprises:
introducing the nucleic acid sequence and a clustered regularly interspaced short palindromic repeats (CRISPR)/nuclease system (CRISPR system) to the cell,
wherein the CRISPR system comprises a guide RNA that directs the nuclease to the region of the gene,
wherein the nuclease cuts at the region of the gene, and
wherein the nucleic acid sequence is inserted at the cut region of the gene for expression.

4. The method of claim 1, wherein the nucleic acid sequence comprises a gene.

5. The method of claim 1, wherein the nucleic acid sequence lacks a promoter.

6. The method of claim 1, wherein the gene comprises myoglobin (MB), and wherein the region comprises a region operable to be expressed by the promoter of MB.

7. The method of claim 1, wherein the region is located at an intron of MB.

8. The method of claim 7, wherein the region comprises a sequence selected from the group consisting of GCCAGACTAGCATCTGGGA (SEQ ID NO: 1), TCCTCTTTAGAAGCCACCA (SEQ ID NO: 2), GGAAGGTATAAAAGCCCTTC (SEQ ID NO: 3), TCTTCTTCAGACTGCGCCA (SEQ ID NO: 4), AGAGCAAGTATGGGCTCACT (SEQ ID NO: 5), TCTTCTTCAGACTGCGCCAT (SEQ ID NO: 6), CTTCTTCAGACTGCGCCATG (SEQ ID NO: 7), GGTCTTCTTCAGACTGCGCCA (SEQ ID NO: 8), AAGGTATAAAAACGCCCTT (SEQ ID NO: 9), a sequence with at least 65% sequence identity with any one of SEQ ID NOS: 1-9, a sequence with at least 70% sequence identity with any one of SEQ ID NOS: 1-9, a sequence with at least 75% sequence identity with any one of SEQ ID NOS: 1-9, a sequence with at least 80% sequence identity with any one of SEQ ID NOS: 1-9, a sequence with at least 85% sequence identity with any one of SEQ ID NOS: 1-9, a sequence with at least 90% sequence identity with any one of SEQ ID NOS: 1-9, a sequence with at least 95% sequence identity with any one of SEQ ID NOS: 1-9, a sequence with at least 99% sequence identity with any one of SEQ ID NOS: 1-9, or combinations thereof.

9. The method of claim 1, wherein the gene comprises creatine kinase (CKM), and wherein the region comprises a region operable to be expressed by the promoter of CKM.

10. The method of claim 9, wherein the region is located at an intron of CKM.

11. The method of claim 10, wherein the region comprises a sequence selected from the group consisting of AGTCCCCAGCAGGATCACAT (SEQ ID NO: 10), TCTGCTCGCAGGGTCCCAA (SEQ ID NO: 11), GCAAGGCTGAGGTTCACAGG (SEQ ID NO: 12), GTGTTACCGAATGGCATGG (SEQ ID NO: 13), TTACCGAATGGCATGGTGG (SEQ ID NO: 14), GGGAAGATTGAAGATTAGC (SEQ ID NO: 15), GGTGTTACCGAATGGCATGG (SEQ ID NO: 16), TACACCGCCACCATGCCATT (SEQ ID NO: 17), GCGGTGTAGGAGACCTGAT (SEQ ID NO: 18), a sequence with at least 65% sequence identity with any one of SEQ ID NOS: 10-18, a sequence with at least 70% sequence identity with any one of SEQ ID NOS: 10-18, a sequence with at least 75% sequence identity with any one of SEQ ID NOS: 10-18, a sequence with at least 80% sequence identity with any one of SEQ ID NOS: 10-18, a sequence with at least 85% sequence identity with any one of SEQ ID NOS: 10-18, a sequence with at least 90% sequence identity with any one of SEQ ID NOS: 10-18, a sequence with at least 95% sequence identity with any one of SEQ ID NOS: 10-18, a sequence with at least 99% sequence identity with any one of SEQ ID NOS: 10-18, or combinations thereof.

12. The method of claim 1, wherein the cell comprises a human cell.

13. The method of claim 1, wherein the cell comprises a skeletal muscle cell.

14. The method of claim 1, wherein the insertion occurs in vitro.

15. The method of claim 1, wherein the insertion occurs in vivo in a subject.

16. The method of claim 15, wherein the expressed nucleic acid sequence is utilized to treat or prevent a disease in the subject.

17. The method of claim 15, wherein the expressed nucleic acid system is utilized for tissue repair or regeneration in the subject.

18. The method of claim 15, wherein the subject is a human being.

19. A system for expressing a nucleic acid sequence in a cell, said system comprising:
a nucleic acid sequence operational for insertion at a region of a gene, wherein the gene comprises a creatine kinase (CKM) gene, a myoglobin (MB) gene, or combinations thereof, and
wherein the inserted nucleic acid sequence is operational to become expressed in the cell by a promoter of the gene.

20. The system of claim 19, wherein the system comprises a clustered regularly interspaced short palindromic repeats (CRISPR)/nuclease system (CRISPR system), wherein the CRISPR system comprises a guide RNA that directs the nuclease to the region of the gene, wherein the nuclease cuts at the region of the gene, and wherein the nucleic acid sequence is inserted at the cut region of the gene for expression.

21. The system of claim 20, wherein the guide RNA directs the nuclease to a region of CKM.

22. The system of claim 21, wherein the guide RNA comprises a sequence that directs the nuclease to a CKM region selected from the group consisting of AGTCCCCAGCAGGATCACAT (SEQ ID NO: 10), TCTGCTCGCAGGGTCCCAA (SEQ ID NO: 11), GCAAGGCTGAGGTTCACAGG (SEQ ID NO: 12), GTGTTACCGAATGGCATGG (SEQ ID NO: 13), TTACCGAATGGCATGGTGG (SEQ ID NO: 14), GGGAAGATTGAAGATTAGC (SEQ ID NO: 15), GGTGTTACCGAATGGCATGG (SEQ ID NO: 16), TACACCGCCACCATGCCATT (SEQ ID NO: 17), GCGGTGTAGGAGACCTGAT (SEQ ID NO: 18), a sequence with at least 65% sequence identity with any one of SEQ ID NOS: 10-18, a sequence with at least 70% sequence identity with any one of SEQ ID NOS: 10-18, a sequence with at least 75% sequence identity with any one of SEQ ID NOS: 10-18, a sequence with at least 80% sequence identity with any one of SEQ ID NOS: 10-18, a sequence with at least 85% sequence identity with any one of SEQ ID NOS: 10-18, a sequence with at least 90% sequence identity with any one of SEQ ID NOS: 10-18, a sequence with at least 95% sequence identity with any one of SEQ ID NOS: 10-18, a sequence with at least 99% sequence identity with any one of SEQ ID NOS: 10-18, or combinations thereof.

23. The system of claim 20, wherein the guide RNA directs the nuclease to a region of MB.

24. The system of claim 23, wherein the guide RNA comprises a sequence that directs the nuclease to an MB region selected from the group consisting of GCCAGACTAGCATCTGGGA (SEQ ID NO: 1), TCCTCTTTAGAAGCCACCA (SEQ ID NO: 2), GGAAGGTATAAAAGCCCTTC (SEQ ID NO: 3), TCTTCTTCAGACTGCGCCA (SEQ ID NO: 4), AGAGCAAGTATGGGCTCACT (SEQ ID NO: 5), TCTTCTTCAGACTGCGCCAT (SEQ ID NO: 6), CTTCTTCAGACTGCGCCATG (SEQ ID NO: 7), GGTCTTCTTCAGACTGCGCCA (SEQ ID NO: 8), AAGGTATAAAAACGCCCTT (SEQ ID NO: 9), a sequence with at least 65% sequence identity with any one of SEQ ID NOS: 1-9, a sequence with at least 70% sequence identity with any one of SEQ ID NOS: 1-9, a sequence with at least 75% sequence identity with any one of SEQ ID NOS: 1-9, a sequence with at least 80% sequence identity with any one of SEQ ID NOS: 1-9, a sequence with at least 85% sequence identity with any one of SEQ ID NOS: 1-9, a sequence with at least 90% sequence identity with any one of SEQ ID NOS: 1-9, a sequence with at least 95% sequence identity with any one of SEQ ID NOS: 1-9, a sequence with at least 99% sequence identity with any one of SEQ ID NOS: 1-9, or combinations thereof.

25. The system of claim 19, wherein the nucleic acid sequence comprises a gene.

26. The system of claim 19, wherein the nucleic acid sequence lacks a promoter.

27. The system of claim 19, wherein the gene comprises myoglobin (MB), and wherein the region comprises a region operable to be expressed by the promoter of MB.

28. The system of claim 27, wherein the region is located at an intron of MB.

29. The system of claim 28, wherein the region comprises a sequence selected from the group consisting of GCCAGACTAGCATCTGGGA (SEQ ID NO: 1), TCCTCTTTAGAAGCCACCA (SEQ ID NO: 2), GGAAGGTATAAAAGCCCTTC (SEQ ID NO: 3), TCTTCTTCAGACTGCGCCA (SEQ ID NO: 4), AGAGCAAGTATGGGCTCACT (SEQ ID NO: 5), TCTTCTTCAGACTGCGCCAT (SEQ ID NO: 6), CTTCTTCAGACTGCGCCATG (SEQ ID NO: 7), GGTCTTCTTCAGACTGCGCCA (SEQ ID NO: 8), AAGGTATAAAAACGCCCTT (SEQ ID NO: 9), a sequence with at least 65% sequence identity with any one of SEQ ID NOS: 1-9, a sequence with at least 70% sequence identity with any one of SEQ ID NOS: 1-9, a sequence with at least 75% sequence identity with any one of SEQ ID NOS: 1-9, a sequence with at least 80% sequence identity with any one of SEQ ID NOS: 1-9, a sequence with at least 85% sequence identity with any one of SEQ ID NOS: 1-9, a sequence with at least 90% sequence identity with any one of SEQ ID NOS: 1-9, a sequence with at least 95% sequence identity with any one of SEQ ID NOS: 1-9, a sequence with at least 99% sequence identity with any one of SEQ ID NOS: 1-9, or combinations thereof.

30. The system of claim 19, wherein the gene comprises creatine kinase (CKM), and wherein the region comprises a region operable to be expressed by the promoter of CKM.

31. The system of claim 30, wherein the region is located at an intron of CKM.

32. The system of claim 31, wherein the CKM region comprises a sequence selected from the group consisting of AGTCCCCAGCAGGATCACAT (SEQ ID NO: 10), TCTGCTCGCAGGGTCCCAA (SEQ ID NO: 11), GCAAGGCTGAGGTTCACAGG (SEQ ID NO: 12), GTGTTACCGAATGGCATGG (SEQ ID NO: 13), TTACCGAATGGCATGGTGG (SEQ ID NO: 14), GGGAAGATTGAAGATTAGC (SEQ ID NO: 15), GGTGTTACCGAATGGCATGG (SEQ ID NO: 16), TACACCGCCACCATGCCATT (SEQ ID NO: 17), GCGGTGTAGGAGACCTGAT (SEQ ID NO: 18), a sequence with at least 65% sequence identity with any one of SEQ ID NOS: 10-18, a sequence with at least 70% sequence identity with any one of SEQ ID NOS: 10-18, a sequence with at least 75% sequence identity with any one of SEQ ID NOS: 10-18, a sequence with at least 80% sequence identity with any one of SEQ ID NOS: 10-18, a sequence with at least 85% sequence identity with any one of SEQ ID NOS: 10-18, a sequence with at least 90% sequence identity with any one of SEQ ID NOS: 10-18, a sequence with at least 95% sequence identity with any one of SEQ ID NOS: 10-18, a sequence with at least 99% sequence identity with any one of SEQ ID NOS: 10-18, or combinations thereof.

33. A modified cell comprising an inserted nucleic acid sequence at a region of a gene, wherein the gene comprises a creatine kinase (CKM) gene, a myoglobin (MB) gene, or combinations thereof, and wherein the inserted nucleic acid sequence becomes expressed in the cell by a promoter of the gene.

34. The modified cell of claim 33, wherein the nucleic acid sequence comprises a gene.

35. The modified cell of claim 33, wherein the nucleic acid sequence lacks a promoter.

36. The modified cell of claim 33, wherein the gene comprises myoglobin (MB), and wherein the region comprises a region operable to be expressed by the promoter of MB.

37. The modified cell of claim 36, wherein the region is located at an intron of MB.

38. The modified cell of claim 37, wherein the region comprises a sequence selected from the group consisting of GCCAGACTAGCATCTGGGA (SEQ ID NO: 1), TCCTCTTTAGAAGCCACCA (SEQ ID NO: 2), GGAAGGTATAAAAGCCCTTC (SEQ ID NO: 3), TCTTCTTCAGACTGCGCCA (SEQ ID NO: 4), AGAGCAAGTATGGGCTCACT (SEQ ID NO: 5), TCTTCTTCAGACTGCGCCAT (SEQ ID NO: 6), CTTCTTCAGACTGCGCCATG (SEQ ID NO: 7), GGTCTTCTTCAGACTGCGCCA (SEQ ID NO: 8), AAGGTATAAAAACGCCCTT (SEQ ID NO: 9), a sequence with at least 65% sequence identity with any one of SEQ ID NOS: 1-9, a sequence with at least 70% sequence identity with any one of SEQ ID NOS: 1-9, a sequence with at least 75% sequence identity with any one of SEQ ID NOS: 1-9, a sequence with at least 80% sequence identity with any one of SEQ ID NOS: 1-9, a sequence with at least 85% sequence identity with any one of SEQ ID NOS: 1-9, a sequence with at least 90% sequence identity with any one of SEQ ID NOS: 1-9, a sequence with at least 95% sequence identity with any one of SEQ ID NOS: 1-9, a sequence with at least 99% sequence identity with any one of SEQ ID NOS: 1-9, or combinations thereof.

39. The modified cell of claim 33, wherein the gene comprises creatine kinase (CKM), and wherein the region comprises a region operable to be expressed by the promoter of CKM.

40. The modified cell of claim 39, wherein the region is located at an intron of CKM.

41. The modified cell of claim 40, wherein the CKM region comprises a sequence selected from the group consisting of AGTCCCCAGCAGGATCACAT (SEQ ID NO: 10), TCTGCTCGCAGGGTCCCAA (SEQ ID NO: 11), GCAAGGCTGAGGTTCACAGG (SEQ ID NO: 12), GTGTTACCGAATGGCATGG (SEQ ID NO: 13), TTACCGAATGGCATGGTGG (SEQ ID NO: 14), GGGAAGATTGAAGATTAGC (SEQ ID NO: 15), GGTGTTACCGAATGGCATGG (SEQ ID NO: 16), TACACCGCCACCATGCCATT (SEQ ID NO: 17), GCGGTGTAGGAGACCTGAT (SEQ ID NO: 18), a sequence with at least 65% sequence identity with any one of SEQ ID NOS: 10-18, a sequence with at least 70% sequence identity with any one of SEQ ID NOS: 10-18, a sequence with at least 75% sequence identity with any one of SEQ ID NOS: 10-18, a sequence with at least 80% sequence identity with any one of SEQ ID NOS: 10-18, a sequence with at least 85% sequence identity with any one of SEQ ID NOS: 10-18, a sequence with at least 90% sequence identity with any one of SEQ ID NOS: 10-18, a sequence with at least 95% sequence identity with any one of SEQ ID NOS: 10-18, a sequence with at least 99% sequence identity with any one of SEQ ID NOS: 10-18, or combinations thereof.

42. The modified cell of claim 33, wherein the cell comprises a human cell.
